(19) United States
(12) Patent Application Publication
(10) Pub. No.: US 2020/0306094 A1
(43) Pub. Date: Oct. 1, 2020

(54) WOUND DRESSING DEVICE, ASSEMBLY AND METHOD

(71) Applicant: REDDRESS LTD., Pardes Hana (IL)

(72) Inventors: Alon KUSHNIR, Givat Ada (IL); Igal KUSHNIR, Pardes Hana (IL)

(73) Assignee: REDDRESS LTD., Pardes Hana (IL)

(21) Appl. No.: 16/649,809

(22) PCT Filed: Sep. 17, 2018
(86) PCT No.: PCT/IL2018/051049

§ 371 Date: Mar. 23, 2020

(30) Foreign Application Priority Data
Sep. 24, 2017 (IL) 254644

(51) Int. Cl. A61F 13/02; A61F 13/00; A61L 15/40; A61L 15/44

(52) U.S. Cl. CPC A61F 13/0216; A61F 13/00068; A61F 13/00063; A61F 13/0246; A61L 2300/418; A61L 15/44; A61F 2013/00417; A61F 2013/00285; A61L 15/40

(57) ABSTRACT

Device, method and assembly for wound treatment. Specifically a wound dressing device; an assembly, e.g. a kit-of-parts, including the device as a component; and method of wound dressing and use wherein the device is a key component. The device includes a cavity defined by concave walls surrounded by lips for attachment to skin in fluid tight manner and a closure removably fixed to the lips and sealing the cavity. The assembly also includes, as another component a device for introducing blood into the cavity after it is fixed over a wound to permit the blood to clot over the wound within the cavity. In use, the clotting mold device is fixed on top of a wound, and blood is introduced into the mold space to permit the blood to clot within the mold space to form a blood clot over the wound.

### TECHNOLOGICAL FIELD

This disclosure is in the field of wound treatment and concerns a wound dressing device, assembly and method.

### BACKGROUND

Chronic wounds and skin ulcers are a serious medical condition and effective wound treatment approaches is a recognized medical need.

U.S. Pat. No. 9,180,142 discloses a wound treatment procedure by which blood is coagulated and the so-formed blood clot is applied onto a wound with a dressing material.

### GENERAL DESCRIPTION

The present disclosure concerns wound treatment through the use of a blood clot. Specifically provided by this disclosure is a wound dressing device and assembly (e.g. in the form of a kit-of-parts for use in the currently disclosed wound treatment) for forming a blood clot, a method for preparing a blood clot-comprising wound dressing, and a method for dressing the wound therewith.

The blood clot that is formed and used according to this disclosure is typically formed from blood of the same subject whose wound is to be dressed by the teaching of this disclosure, withdrawn from the subject in any manner acceptable in medical practice for blood withdrawal. However, the use of blood from a different source, e.g. blood obtained from a blood bank, is also contemplated in accordance with this disclosure.

Three aspects are provided by this disclosure: one concerns a wound dressing device; the other concerns an assembly, e.g. in the form of a kit-of-parts, which comprises said device as one of its components; and the third concerns a method of wound dressing and a use wherein said device is a key component.

All three aspects center around the formation of a blood clot, in situ, onto a wound, as part of the wound dressing procedure. In other words, the blood, still in liquid state, is brought into contact with the wound and is induced to clot while at least partially in contact with the wound to be dressed therewith. All three aspects are encompassed in the following description by the term “wound dressing procedure”.

The method comprises fixing a device of the kind disclosed herein, on top of the wound, the device defining with the wound an enclosed space which serves as a mold (cast) for clotting blood on top of the wound. This space will be referred to herein as a “mold space”.

The device comprises a cavity or depression surrounded by lips configured for attachment to skin in a fluid tight manner. The cavity or depression may have walls that may be generally concave, polygonal or any other suitable shape. In some embodiments, the cavity is shallow, namely, it has a depth relatively smaller than the overall area confined by the walls.

The lips typically define narrow flat surfaces that when brought into contact with the skin, should be relatively level and smooth to permit fluid tight attachment to the skin. By an embodiment of this disclosure, the lips may carry an adhesive that will ensure fluid tight attachment to the skin.

In addition, in some embodiments, the adhesive may be applied onto the skin or onto the lips, prior to attachment. At times, according to this embodiment, at least a portion of the lips may have a rough surface onto which the adhesive is applied prior to fixation onto the skin. It is also possible, by other embodiments of this disclosure, to fit a two-sided adhesive strip onto the lips or the skin to be used as means for attachment. By still another embodiment, the attachment may be by means of an adhesive tape fitted over the device's lips and the surrounding skin portions for tight association throughout the blood clot formation process.

By other embodiments, the attachment of the lips to the skin may be by forming a vacuum within the mold space, by forcing the device (and hence the lips) against the skin by hand, by the use of an elastic band, adhesive tape or other means for tight forcing of the device against the skin.

The wound's surface area confined between the lips may have different shapes and sizes to suit wounds of different shapes and sizes. Thus, the wound dressing procedure may also involve selection of a device of the proper shape and size to permit the lips to be placed on skin portions surrounding the wound's boundaries. In some embodiments, the device is flexible or pliable to permit its shaping and/or stretching to a desired shape and wound surface area to be confined between the lips.

The device may also comprises a closure that is removably attached to the lips and seals the cavity until use. The closure may be useful for maintaining sterility of the cavity and for holding elements, such as a coagulant initiator and/or scaffold matrix within the cavity. The closure may, for example, be a film made of plastic, foil, a combination thereof, etc. that is pealed from the opening of the cavity (the lips) prior to fixation onto the skin.

according to the wound dressing procedure disclosed herein, once the lips are firmly fixed to skin portions surrounding the wound, a mold space is formed, which, as noted above, is defined between the surface of the wound and the cavity's walls. Blood, typically whole blood, can then be introduced into the mold space and permitted to clot within the mold space to form a blood clot over the wound. The blood clot is maintained over the wound for a time period. This time period may vary and is typically several hours, several days or several weeks, e.g. 1 day, 2-6 days, 1 week, 2-4 weeks, and at times even longer, as well as for any period of time in-between than that indicated. In some embodiments, the blood clot may be maintained over the wound for the entire healing process of several weeks to several month. In some embodiments the wound treatment comprises periodical refreshing procedures that comprises removal of an existing blood clot from the wound and then forming a new blood clot on the wound in a manner as described above. The time period between a blood clot formation over a wound and the performance of a refreshing procedure may be, for example, 1 day, 2-6 days, 1 week, 2-4 weeks, etc., as well as for any period of time in-between than that indicated. The entire wound healing procedure may involve several consecutive refreshing procedures.

The device's walls may be made from a variety of materials. Typically, in order to permit monitoring the blood introduction and the clot formation, the walls or parts thereof are transparent.

Introduction of the blood involves, by one embodiment, piercing the wall with a needle and then injecting blood into the mold space through the needle. Optionally, this introduction is preceded by a second piercing of the wall on an adjacent or different location, to thereby form a vent aperture to permit release of excess pressure during blood introduction. By other embodiments, the walls may comprise a venting valve which, a priori, is sealed and opened before the blood introduction and/or a port for introducing the blood into the mold space.

By one embodiment, the device is removed after the blood clot is formed. To facilitate easy removal, e.g. without severing the integrity of the formed blood clot, the walls are preferably made of or coated with a material to which a blood clot does not adhere. By another embodiment, the device or part thereof remains in situ after the blood clot is formed and serves as part of the wound dressing.

In the case where the device is removed after clot formation, a dressing material, for example, gauze, plaster or bandage, may be fitted over the blood clot to protect and secure the blood clot in place. The wound dressing procedure, by some embodiments, may make use of a scaffold matrix combined with and brought into contact with the blood prior to its clotting, so as to become integrated with the thus formed blood clot. The scaffold matrix supports the clot and assists in maintaining the clot's structural integrity. The scaffold matrix may, by one embodiment, be an independent element fitted onto the wound, prior to said fixing of the device onto the skin; by another embodiment, the scaffold matrix may be comprised within the cavity, e.g. a priori comprised therein or introduced into the cavity before fixing the device onto the wound. The scaffold matrix may, for example, be a 2-dimensional or 3-dimensional matrix, may be a porous material, may have a netlike structure; may be made of a polymeric material such as plastic, may be made out of natural or synthetic fibers or made out of a woven or non-woven cloth, e.g. gauze.

In order to ensure controlled coagulation, the blood may be contacted with one or combination of coagulation initiators, namely, one or more substances (synthetic or naturally occurring) that promotes/activates blood coagulation. In some embodiments, the coagulation initiator is kaolin. When using a coagulation initiator, coagulation of blood may involve, for example, mixing the blood with kaolin shortly before introduction into the mold space. Alternatively, the coagulation initiator may a priori be present within the cavity; for example, attached to the walls or incorporated into a scaffold matrix, placed within or a priori comprised within the cavity or further, separately introduced into the mold space (e.g. by injection) before or after introduction of the blood. In addition or alternatively, the coagulation initiator may be incorporated into a scaffold matrix that is fitted onto the wound, prior to fixing of the device over the found.

In some embodiments, the coagulation initiator is independently stored, e.g. as a powder, granulate or liquid in a separate container and subsequently mixed with the blood upon introduction into the mold space or applied onto the scaffold matrix prior to or after introducing the blood. By still another alternative, the coagulation initiator may be applied directly onto the wound as a first step in the wound treatment procedure.

The wound dressing assembly or kit of this disclosure comprises a clotting mold device of the kind specified above. In addition, according to some embodiments, the assembly or kit may comprise other elements or devices required for the in situ formation of the blood clot on the wound, e.g. a syringe for introducing blood into the cavity. The assembly or kit may comprise also a dressing material for applying over the blood clot formed over the wound.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows a wound dressing device according to an embodiment of this disclosure.

FIG. 2 shows the wound dressing device after removal of the closure and applied onto the skin.

FIGS. 3A and 3B show, respectively, two steps of introducing blood into the mold space.

FIG. 4 shows the wound dressing device with blood still in liquid form with the device secured by adhesive tape to the skin.

FIG. 5 shows the blood clot with the device partially ruptured prior to removal.

FIG. 6 shows the blood clot on the skin after its formation and after the device has been removed.

### DETAILED DESCRIPTION OF EMBODIMENTS

The invention will now be illustrated with reference to embodiments shown in the annexed Figures. It should be noted that these Figures are pictures from a test trial on skin without a wound, to demonstrate the wound dressing procedure of this disclosure.

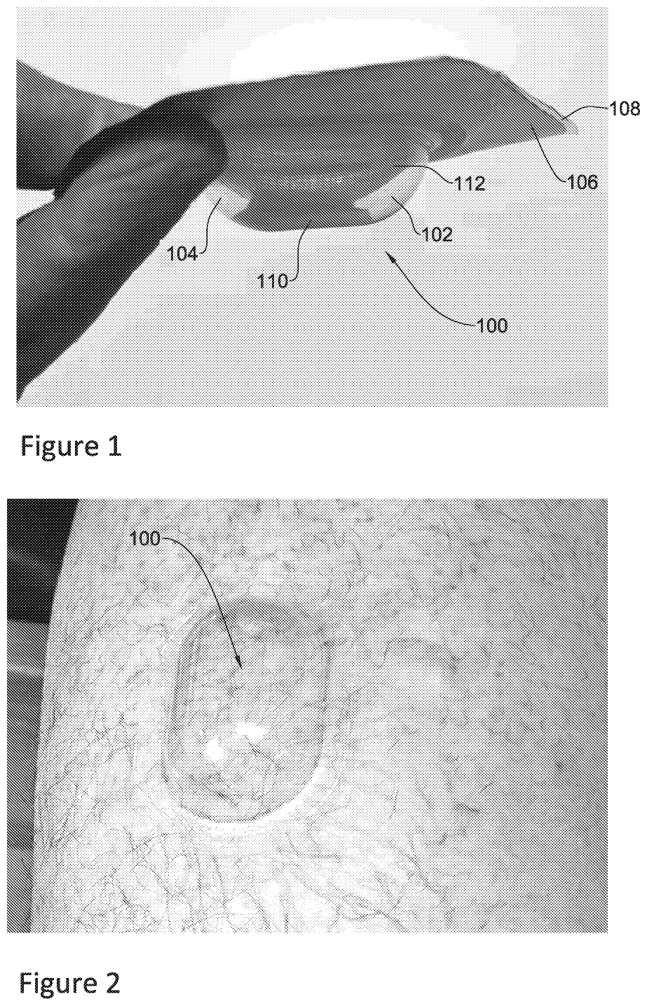

The device 100 shown in FIG. 1 includes generally concave walls 102 that form a cavity 104 defined between lips 106 that are generally flat. The opening defined between the lips is sealed by a removable closure 108 which is a laminate/film fitted onto lips 106. After removal of closure 108, the device 100 is placed onto skin, an adhesive remaining after removal of the closure causes it to adhere to the skin. A device 100 fixed to a subject's skin is shown in FIG. 2.

Also shown in FIG. 1 is a scaffold matrix 112 placed within the cavity, the scaffold matrix holds a coagulation initiator 110.

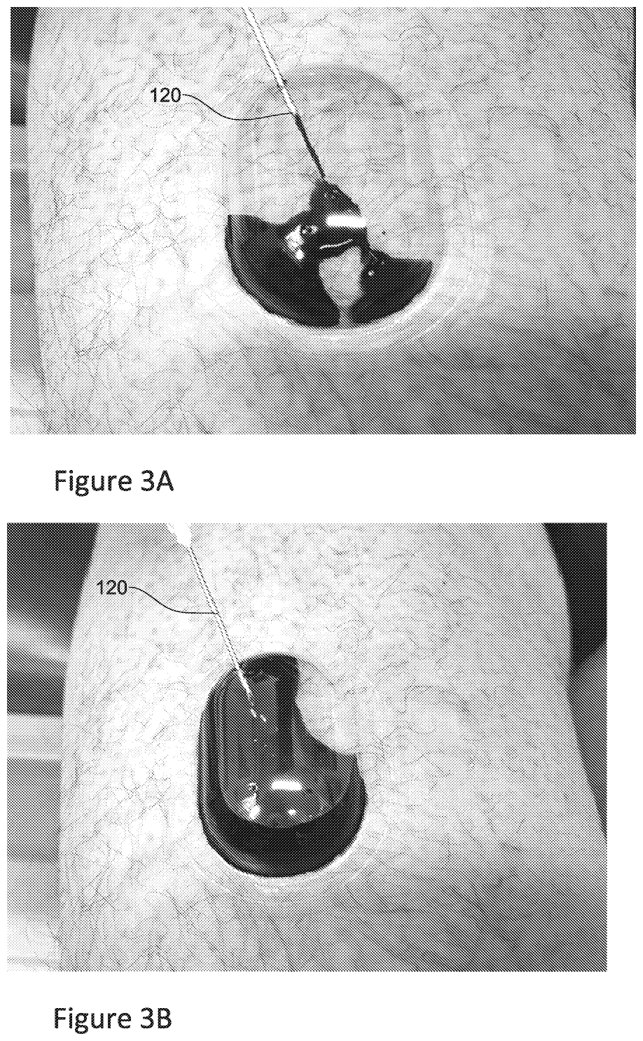

Turning now to FIGS. 3A-3B, steps of introducing blood, typically whole blood into the mold space are shown. Specifically, blood withdrawn from the subject or alternatively taken from a blood bank, is injected with a needle 120 into the mold space formed between the walls 102 and underlying portion of the skin or wound. According to this non-limiting embodiment, upon introduction into the mold space, the blood is mixed with the coagulation initiator 110 a priori present in the cavity. The needle 120 first pierces the walls of the device and then injects the blood contained in the syringe (not seen).

In some other embodiments, the blood is pre-mixed with a coagulation initiator, such as Kaolin, that is provided in a separate container (not shown), or the coagulation initiator is introduced into the mold space separately, either before, during or after the blood introduction.

As can also be seen in FIGS. 1-3B the walls of the device are transparent to permit monitoring the introduction and subsequent blood clotting.

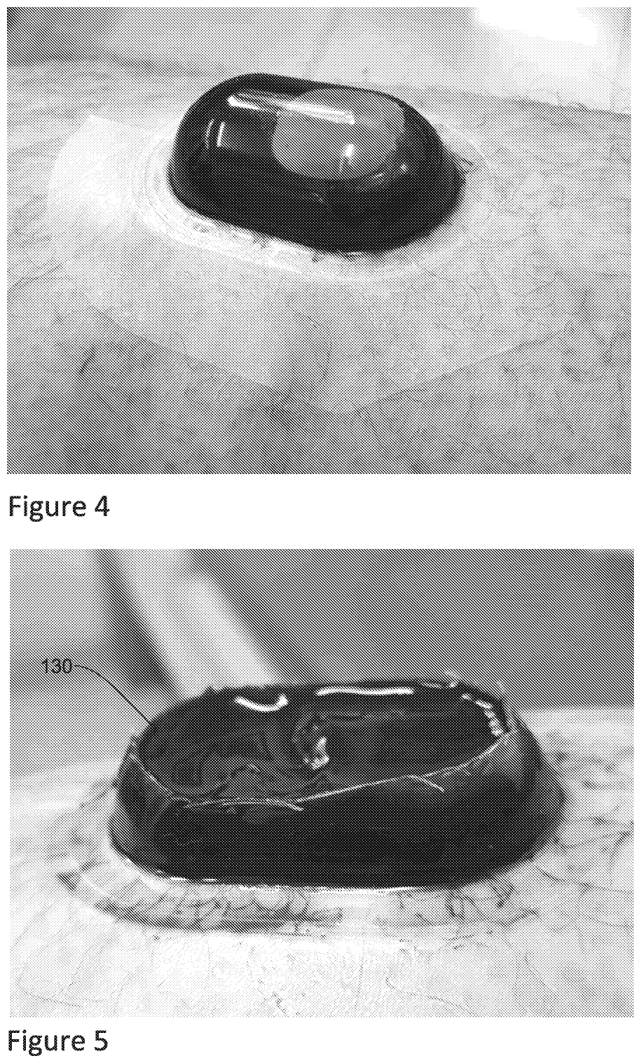

The device is then allowed to remain attached to the skin with the blood held within the mold space for a time at least until the blood has clotted. Such time may range from several days, to one or more weeks, The device can be secured to the skin by use of an adhesive strip 130, as seen in FIG. 4.

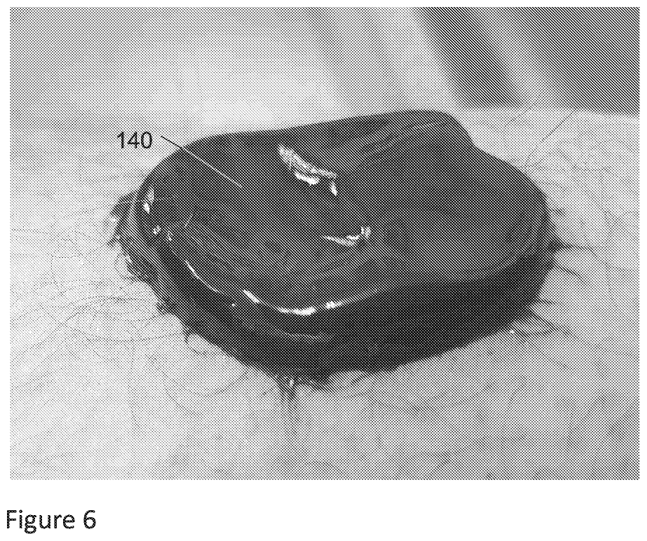

After a period of time, a blood clot is formed and the walls of the device can then be cut away, as seen in FIG. 5; and, after removal, a blood clot 140 remains on top of the skin, as seen in FIG. 6. Where the procedure will be performed over a wound, the blood clot will cover the wound and a dressing material, which may be conventional dressing material, such as gauze or bandage, will be applied over the clot for protecting the wound and securing the clot in place.

1.-8. (canceled)

9. A method for dressing a wound, comprising:
fixing a clotting mold device on top the wound,
said device comprises a cavity surrounded by lips that are configured for attachment to skin in a fluid tight manner, and
said fixing comprises attaching the lips to skin portions surrounding the wound to thereby form a mold space defined between the wound and the cavity;
introducing blood into the mold space and permitting the blood to clot within the mold space to thereby form a blood clot over the wound; and
maintaining the blood clot over the wound for a wound-healing period.

10. The method of claim 9, wherein said fixing comprise adhering the lips onto said skin portions.

11. The method of claim 10, wherein the lips comprises said adhesive or said adhesive is fitted onto said lips or onto said skin portions prior to said fixing.

12. The method of claim 9, wherein said fixing comprises forming a vacuum within the mold space.

13. The method of claim 9, comprising combining the blood clot with a scaffold matrix prior to blood clot formation, such that said scaffold matrix becomes integrated within the formed blood clot.

14. The method of claim 13, wherein said scaffold matrix is fitted onto the wound prior to said fixing.

15. The method of claim 14, wherein said scaffold matrix is comprised within said cavity.

16. The method of claim 9, comprising
following formation of the blood clot, removing said device off the wound; and
fixing a dressing material, e.g. gauze, over the blood clot.

17. The method of claim 9, wherein said introducing comprises:
piercing the walls by a needle; and
injecting blood into said mold space through the needle.

18. The method of claim 9, wherein the blood is whole blood.

19. The method of claim 9, comprising contacting the blood with a coagulation initiator, said coagulation initiator is characterized by one of the following:
is mixed with the blood prior to said introducing,
is a priori present in the cavity,
is added into said mold space prior to or after said introducing, and
is incorporated into a scaffold matrix that is comprised within said cavity or fitted onto the wound prior to said fixing.

20. (canceled)

21. The method of claim 9, wherein said device comprises a closure, removably fixed to the lips and sealing the cavity, said closure is removed prior to said fixing.

22. A wound dressing assembly, comprising
a clotting mold device comprising a cavity defined by concave walls surrounded by lips configured for attachment to skin in a fluid tight manner, wherein, for introducing blood into the cavity once said lips are attached to the skin, the concave walls of the clotting mold device comprise one of (i) a portion being pierceable and suitable for introducing blood into the cavity, and (ii) a port suitable for introducing said blood into the cavity,
a device for introducing blood into the cavity after it is fixed over a wound to permit the blood to come into contact with the wound and to clot over the wound within said cavity; and
a blood coagulation initiator.

23. The wound dressing assembly of claim 22, wherein the device comprises a closure removably fixed to the lips and sealing the cavity.

24.-26. (canceled)

27. The wound dressing assembly of claim 22, comprising a scaffold matrix configured for integration within the formed blood clot.

28. The wound dressing of assembly of claim 27, wherein the scaffold matrix is comprised within said cavity.

29. The wound dressing assembly of claim 22, comprising one or both of
a syringe and a needle for piercing the walls and injection of the blood into the cavity, and
a dressing material for fixing over the blood clot.

30. The wound dressing assembly of claim 22, wherein said coagulation initiator
is in a form of a powder, granulate or solution suitable for mixing with the blood or for applying directly onto the wound,
being a priori present in the cavity,
being incorporated into a scaffold matrix that is comprised within said cavity or intended for placement onto the wound prior to said attachment.

31. The wound dressing assembly of claim 22, wherein the walls of the cavity comprise one or both of
a vent that is a priori sealed, for removal of excess pressure during introduction of the blood into the enclosure, and
a port for introducing blood into the mold space.

32. The wound dressing assembly of claim 22, wherein the enclosure is made of a material to which a blood clot does not adhere.

33.-34. (canceled)
